SEQUENCE LISTING (38 sequences)

SEQ ID NO: 1 - DNA, 20 bp, Artificial Sequence
  Description of Artificial Sequence: Synthetic primer
ccagcagccgcggtaatacg

SEQ ID NO: 2 - DNA, 23 bp, Artificial Sequence
  Description of Artificial Sequence: Synthetic primer
tacggttaccttgttacgacttc

SEQ ID NO: 3 - DNA, 122 bp, Artificial Sequence
  Description of Artificial Sequence: Synthetic polynucleotide
ctccaagtcgacatcgtttacggtctctgctgcttcagcttcccagtcgtggtagtacatccatcgtggtacatacgagcgatatccgacggtagtgtacccccgtcaattcatttgagttt

SEQ ID NO: 4 - DNA, 117 bp, Artificial Sequence
  Description of Artificial Sequence: Synthetic polynucleotide
ctggaattctacccccctctacgtctctgctgcttcagcttcccagtcgtggtagtacatccatcgtggtacatacgagcgatatccgacggtagtgtaccacaacacgagctgacg

SEQ ID NO: 5 - DNA, 124 bp, Artificial Sequence
  Description of Artificial Sequence: Synthetic polynucleotide
ccgtattaccgcggctgctggtctctgctgcttcagcttcccagtcgtggtagtacatccatcgtggtacatacgagcgatatccgacggtagtgtacccctacggttaccttgttacgacttc

SEQ ID NO: 6 - DNA, 17 bp, Artificial Sequence
  Description of Artificial Sequence: Synthetic primer
atccgacggtagtgtac

SEQ ID NO: 7 - DNA, 17 bp, Artificial Sequence
  Description of Artificial Sequence: Synthetic primer
agctgaagcagcagaga

SEQ ID NO: 8 - DNA, 21 bp, Artificial Sequence
  Description of Artificial Sequence: Synthetic probe
gagtattaccgcggcgaattc

SEQ ID NO: 9 - DNA, 37 bp, Artificial Sequence
  Description of Artificial Sequence: Synthetic probe
aacacttcttgcggcgatggttcctggctcttcgatc

SEQ ID NO: 10 - DNA, 23 bp, Artificial Sequence
  Description of Artificial Sequence: Synthetic probe
agagaagtcctagcacggtaacc

SEQ ID NO: 11 - DNA, 44 bp, Artificial Sequence
  Description of Artificial Sequence: Synthetic primer
agagaagtcctagcacggtaacctccgaggatgtcatcaaagag

SEQ ID NO: 12 - DNA, 35 bp, Artificial Sequence
  Description of Artificial Sequence: Synthetic primer
aagctggaattcgcttccgtactggaactgagggc

SEQ ID NO: 13 - DNA, 33 bp, Artificial Sequence
  Description of Artificial Sequence: Synthetic primer
aagctggaattcatgacagggccatcggagggg

SEQ ID NO: 14 - DNA, 242 bp, Artificial Sequence
  Description of Artificial Sequence: Synthetic polynucleotide
agagaagtcctagcacggtaacctccgaggatgtcatcaaagagtttaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcgaattccagctt

SEQ ID NO: 15 - DNA, 442 bp, Artificial Sequence
  Description of Artificial Sequence: Synthetic polynucleotide
agagaagtcctagcacggtaacctccgaggatgtcatcaaagagtttaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgaattccagctt

SEQ ID NO: 16 - DNA, 21 bp, Artificial Sequence
  Description of Artificial Sequence: Synthetic primer
gagtattaccgcggcgaattc

SEQ ID NO: 17 - DNA, 18 bp, Artificial Sequence
  Description of Artificial Sequence: Synthetic primer
  Description of Combined DNA/RNA Molecule: Synthetic primer
  misc_feature (1)..(4): Phosphorothioate bond between nucleotides
atcgccgcaagaagtgtu

SEQ ID NO: 18 - DNA, 19 bp, Artificial Sequence
  Description of Artificial Sequence: Synthetic primer
ggttcctggctcttcgatc

SEQ ID NO: 19 - skipped

SEQ ID NO: 20 - DNA, 155 bp, Artificial Sequence
  Description of Artificial Sequence: Synthetic probe
gagtattaccgcggcgaattcatgagccatattcaacgggaaacgtcttgctctaggaacacttcttgcggcgatagaaggttcctggctcttcgatcgcagtttcatttgatgctcgatgagtttttctaaagagaagtcctagcacggtaacc

SEQ ID NO: 21 - DNA, 132 bp, Artificial Sequence
  Description of Artificial Sequence: Synthetic probe
gagtattaccgcggcgaattcccaacggcagcagcggatccgtgaacacttcttgcggcgatagaaggttcctggctcttcgatctgatttatggtcattctcgttttcagagaagtcctagcacggtaacc

SEQ ID NO: 22 - DNA, 145 bp, Artificial Sequence
  Description of Artificial Sequence: Synthetic probe
gagtattaccgcggcgaattcttggagtttgcttccggtctggttcgcaacacttcttgcggcgatagaaggttcctggctcttcgatcgatttgggtaatgaatatccggttcttgtcaagagagaagtcctagcacggtaacc

SEQ ID NO: 23 - DNA, 137 bp, Artificial Sequence
  Description of Artificial Sequence: Synthetic probe
gagtattaccgcggcgaattcttggagtttgcttccggtctggttcgcaacacttcttgcggcgatagaaggttcctggctcttcgatcgccgttgctaccctcgttccgatgcagagaagtcctagcacggtaacc

SEQ ID NO: 24 - DNA, 137 bp, Artificial Sequence
  Description of Artificial Sequence: Synthetic probe
gagtattaccgcggcgaattcttggagtttgcttccggtctggttcgcaacacttcttgcggcgatagaaggttcctggctcttcgatcggctctgagggtggcggttctgaggagagaagtcctagcacggtaacc

SEQ ID NO: 25 - DNA, 138 bp, Artificial Sequence
  Description of Artificial Sequence: Synthetic probe
gagtattaccgcggcgaattcttggagtttgcttccggtctggttcgcaacacttcttgcggcgatggttcctggctcttcgatcggcgaatccgttattgtttctcccgatgtaagagaagtcctagcacggtaacc

SEQ ID NO: 26 - DNA, 29 bp, Artificial Sequence
  Description of Artificial Sequence: Synthetic primer
caaaccgctaagctcaaggtcacaaaagg

SEQ ID NO: 27 - DNA, 30 bp, Artificial Sequence
  Description of Artificial Sequence: Synthetic primer
cgcttccctccatcttgaccttaaatctca

SEQ ID NO: 28 - DNA, 25 bp, Artificial Sequence
  Description of Artificial Sequence: Synthetic primer
gtgaaactcagaggaaccaacttcc

SEQ ID NO: 29 - DNA, 30 bp, Artificial Sequence; identical to SEQ ID NO: 27
  Description of Artificial Sequence: Synthetic primer

SEQ ID NO: 30 - DNA, 35 bp, Artificial Sequence
  Description of Artificial Sequence: Synthetic primer
tcctctgagtttcaccggatccgcgacccatttgc

SEQ ID NO: 31 - DNA, 38 bp, Artificial Sequence
  Description of Artificial Sequence: Synthetic primer
tcaagatggagggaagcgaattcgagctccgtcgacaa

SEQ ID NO: 32 - DNA, 15 bp, Artificial Sequence
  Description of Artificial Sequence: Synthetic oligonucleotide
ttatcctggatatcg

SEQ ID NO: 33 - DNA, 15 bp, Artificial Sequence
  Description of Artificial Sequence: Synthetic oligonucleotide
ggatagctagatgac

SEQ ID NO: 34 - DNA, 15 bp, Artificial Sequence
  Description of Artificial Sequence: Synthetic oligonucleotide
acttatggacctagc

SEQ ID NO: 35 - DNA, 15 bp, Artificial Sequence
  Description of Artificial Sequence: Synthetic oligonucleotide
cctggatcttggatt

SEQ ID NO: 36 - DNA, 15 bp, Artificial Sequence
  Description of Artificial Sequence: Synthetic oligonucleotide
attgactaattaccg

SEQ ID NO: 37 - DNA, 55 bp, Artificial Sequence
  Description of Artificial Sequence: Synthetic oligonucleotide
atgaattcatgcgtgctttaccgatctgtttagtagcactcatgctaagcggctg

SEQ ID NO: 38 - DNA, 60 bp, Artificial Sequence
  Description of Artificial Sequence: Synthetic oligonucleotide
ctatatttcgccgtaatgttaagcgcagaacgatacatacagcctgacgatagcagcctg

(19) United States
(12) Patent Application Publication
(10) Pub. No.: US 2018/0171386 A1
(43) Pub. Date: Jun. 21, 2018

(54) Long Adapter Single Stranded Oligonucleotide (LASSO) Probes to Capture and Clone Complex Libraries

(71) Applicants: The General Hospital Corporation, Boston, MA (US); The Scripps Research Institute, La Jolla, CA (US)

(72) Inventors: Biju Parekkadan, Cambridge, MA (US); Lorenzo Tosi, Watertown, MA (US); Harry Benjamin Larman, Baltimore, MD (US)

(21) Appl. No.: 15/579,136

(22) PCT Filed: Jun. 3, 2016
(86) PCT No.: PCT/US16/35919

§ 371 Date: Dec. 1, 2017

Related U.S. Application Data
(60) Provisional application No. 62/170,648, filed on Jun. 3, 2015.

(51) Int. Cl. C12Q 1/6806

(52) U.S. Cl. CPC C12Q 1/6806

(57) ABSTRACT

Long adapter single strand oligonucleotide (LASSO) probes that can be used to capture and clone thousands of kilobase-sized DNA fragments in a single reaction, as well as methods of generating the same.

### CLAIM OF PRIORITY

This application claims the benefit of U.S. Provisional Application Ser. No. 62/170,648, filed on Jun. 3, 2015. The entire contents of the foregoing are incorporated herein by reference.

### FEDERALLY SPONSORED RESEARCH OR DEVELOPMENT

This invention was made with Government support under Grant Nos. R01EB012521 and K01DK087770 awarded by the National Institutes of Health. The Government has certain rights in the invention.

### TECHNICAL FIELD

Described herein are long adapter single strand oligonucleotide (LASSO) probes that can be used to capture and clone thousands of kilobase-sized DNA fragments in a single reaction.

### BACKGROUND

The ability to isolate or enrich specific genomic loci for downstream analyses has transformed our understanding of molecular and cellular biology (Turner et al., Annu Rev Genomics Hum Genet 10, 263-284 (2009)).

### SUMMARY

Molecular inversion probes (MIPs) are single stranded DNA molecules that become circularized by gap filling after annealing to target sequences that flank a desired DNA fragment. MIPs have proven to be a useful tool for target capture, since they exhibit high specificity and can be massively multiplexed (Turner et al., Nat Methods 6, 315-316 (2009)). However, the ability of traditional MIPs to capture target sequences greater than ˜200 bp is precluded by constraints associated with the physical bending of DNA. Described herein are long adapter single strand oligonucleotide (LASSO) probes that can be used to capture and clone thousands of kilobase-sized DNA fragments in a single reaction. More than 3000 bacterial open reading frames were simultaneously cloned from genomic DNA (spanning 400-5,000 bp sized targets) in just 2 hours. This present technology enables long-read sequencing library preparation and massively parallel cloning.

Thus, described herein are Long Adapter Single Stranded Oligonucleotides (LASSOS) comprising, from 5′ to 3′:

a ligation arm sequence of 20-40, 15-80, nucleotides (nt) complementary to a 5′ region of a target sequence (i.e., a single contiguous target sequence, e.g., a genomic sequence, lncRNA, cDNA or other);
a Long Adapter sequence of 200 to 2500 nt, e.g., 200-500, 200-2000, 200-2500, 200-1500, 200-1000, or 200-800 nt, preferably 250-300 nt, comprising a fusion overlapping sequence and optionally one or more restriction enzyme recognition sites;
an extension arm sequence that is 15-80 nt, preferably 20-40 nt long, complementary to a 3′ region of a target sequence,
wherein the ligation arm and extension arm sequences are complementary to 5′ and 3′ regions of a single target sequence and the complementary regions are at least 200-30,000 nts apart, e.g., at least 500, 1000, 5,000, 10,000, 20,000, or 30,000 nt apart on the target sequence, and wherein the Long Adapter sequence is not complementary to the target sequence.

In some embodiments, the target sequence is a coding or noncoding DNA sequence including complete or partial open reading frames, complete or partial intronic DNA regions or other noncoding sequence such as lincRNA or regulatoryRNA. The target sequence can also optionally be from a sample of gDNA or cDNA, e.g., from prokaryotic (g/c)DNA or a eukaryotic (g/c)DNA found within (e.g., mitochrondria, stool, tissue lysate, cell lysate, sputum, blood serum/plasma, bone marrow, saliva, or tissue swab).

Also provided herein are pluralities of the LASSO oligonucleotides, wherein the plurality includes oligonucleotides with sequences complementary to 10 or more, 100 or more, 1000 or more, 10,000 or more, 100,000 or more, or 100,000,000 or more different target sequences.

In addition, provided herein are pluralities of pre-LASSO probes, preferably wherein the pre-LASSO probes are synthetically generated, preferably 80-200 base pairs (bp) long, comprising (i) a ligation arm sequence of 15-80 bp, preferably 20-40 bp long, that is complementary to a 5′ region of a target sequence, (ii) an extension arm sequence of 15-80 bp, preferably 20-40 bp long, that is complementary to a 3′ region of a target sequence, wherein the ligation arm and extension arm sequences are complementary to 5′ and 3′ regions of a single target sequence and the complementary regions are at least 200-30,000 nts apart, e.g., at least 500, 1000, 5,000, 10,000, 20,000, or 30,000 nt apart on the target sequence, (iii) primer annealing sites, preferably 15-40 bp long, at the 5′ end of the pre-LASSO probes and between the ligation arm and extension arm sequences, and (iv) a fusion overlapping sequence, preferably 15-50 bp long, at the 3′ end of the pre-LASSO probes, wherein the plurality of pre-LASSO probes comprises probes with sequences complementary to 10 or more, 100 or more, 1000 or more, 10,000 or more, 100,000 or more, or 100,000,000 or more different target sequences, preferably wherein all or a subset of the pre-probes have the same primer annealing site sequences and fusion overlapping sequences.

Further, described herein are methods for generating the plurality of oligonucleotides of claim 1. The methods can include

(i) providing a plurality of pre-LASSO probes preferably wherein the pre-LASSO probes are synthetically generated, preferably 80-200 base pairs (bp) long, comprising (i) a ligation arm sequence of 15-80 bp, preferably 20-40 bp long, that is complementary to a 5′ region of a target sequence, (ii) an extension arm sequence of 15-80 bp, preferably 20-40 bp long, that is complementary to a 3′ region of a target sequence, wherein the ligation arm and extension arm sequences are complementary to 5′ and 3′ regions of a single target sequence and the complementary regions are at least 200-30,000 nts apart, e.g., at least 500, 1000, 5,000, 10,000, 20,000, or 30,000 nt apart on the target sequence, (iii) primer annealing sites, preferably 15-40 bp long, at the 5′ end of the pre-LASSO probes and between the ligation arm and extension arm sequences, and (iv) a fusion overlapping sequence, preferably 15-50 bp long, at the 3′ end of the pre-LASSO probes, wherein the plurality of pre-LASSO probes comprises probes with sequences complementary to 10 or more, 100 or more, 1000 or more, 10,000 or more, 100,000 or more, or 100,000,000 or more different target sequences, preferably wherein all or a subset of the pre-probes have the same primer annealing site sequences and fusion overlapping sequences;

(ii) contacting the plurality of pre-LASSO probes with a plurality of Long Adapter Oligonucleotides in a single reaction sample, wherein the Long Adapter Oligonucleotides comprise a sequence of 200 to 2500 nt, e.g., 200-500, 200-2000, 200-2500, 200-1500, 200-1000, or 200-800 nt, preferably 250-300 nt, comprising a fusion overlapping sequence that is complementary to the fusion overlapping sequence on the pre-LASSO probes, a primer annealing site of 15-80 nts, optionally one or more restriction enzyme recognition sites and a long adapter sequence, under conditions to allow hybridization of the fusion overlapping sequences of the long adapters to the pre-probes at the fusion overlapping sequence;

(iii) using overlap-extension polymerase chain reaction (PCR) to extend the hybridized regions to generate a double stranded linear DNA fragment;

(iv) digesting the double-stranded linear DNA fragment to create complementary overhangs or blunt ends to allow circularization of the double-stranded DNA fragment;

(v) circularizing the double-stranded DNA fragment by enzymatic and/or chemical ligation; and

(vi) using inverted PCR with primers that bind to the primer annealing sites between the ligation arm and extension arm sequences to create linear double-stranded DNA fragments with the primer annealing sites at the 5′ and 3′ ends of linear double-stranded DNA fragments; and

(viii) removing all or part of the primer annealing sites from the 5′ and 3′ ends of linear oligonucleotides by restriction digestion and/or glycosylase digestion.

In addition, provided herein are methods for creating a library of target sequences, e.g., 10 or more, 100 or more, 1000 or more, 10,000 or more, 100,000 or more, or more different target sequences, from a sample. The methods can include contacting the sample with the plurality of the oligonucleotides of claim 3 in a single reaction sample, wherein the plurality includes oligonucleotides with sequences complementary to the different target sequences, under conditions sufficient to allow hybridization of the ligation arm and extension arm sequences of the oligonucleotides to target sequences in the sample;

gap filling using polymerase and ligase to copy the target sequence between the ligation arm and extension arm and ligate the resulting molecule, to create circular single-stranded DNA fragments comprising the target sequences;
purifying the circular single-stranded DNA fragments comprising the target sequences, optionally by digesting linear DNA in the sample; and
amplifying the circular single-stranded DNA fragments comprising the target sequences, thereby amplifying the target sequences.

In some embodiments, the target sequences are at least 200-500 base pairs (bp) long.

In some embodiments, the target sequences are at least 200-30,000 long, e.g., at least 500, 1000, 5,000, 10,000, 20,000, or 30,000 bp long.

In some embodiments, gap filling using polymerase and ligase comprises using 0.03-0.05, e.g., 0.04, U/μl polymerase and 0.02-0.1, e.g., 0.025, U/μl thermostable ligase.

In some embodiments, hybridization of the ligation arm and extension arm sequences of the oligonucleotides to target sequences, and gap filling were performed at 55-75° C., preferably at 65° C.

In some embodiments, the target sequences comprise 10,000 or more different target sequences.

In some embodiments, the sample is a genomic DNA (gDNA) sample or comprises cDNA. The target sequence can also optionally be from a sample of gDNA or cDNA, e.g., from prokaryotic (g/c)DNA or a eukaryotic (g/c)DNA found within (e.g., mitochrondria, stool, tissue lysate, cell lysate, sputum, blood serum/plasma, bone marrow, saliva, or tissue swab).

Further, provided herein are libraries of target sequences created by a method described herein.

In addition, described herein are kits for use in a method described herein, e.g., comprising one or more of the LASSO or pre-LASSO probes described herein, and optionally one or more additional reagents for performing the methods described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Exemplary Synthesis of DNA LASSO Probes. (1A) Exemplary schematic of a final ssDNA LASSO probe. Two sequences complementary to regions that flank a target are linked to a universal adapter by a series of processing reactions. (1B) Schematic of starting components for LASSO probe synthesis, consisting of pre-LASSO probe and a Long Adapter. (1C) Exemplary Schematic of PCR reaction used to fuse the Long Adapter and pre-LASSO probe. Gel electrophoresis results illustrate successful fusion. Lanes: 1: Long Adapter (220 bp); 2: Pre-LASSO probe (125 bp); 3: Fused product (345 bp); Ladder: Quick-Load 100 bp. (1D) Schematic of a intramolecular circularization reaction of the fusion PCR product. Not shown is the subsequent digestion of residual linear DNA. Gel electrophoresis results illustrate successful, ligation-dependent circularization. Lanes: 1: Circular Product (550 bp); 2: Linearized Product (550 bp); 3: No Ligase Digestion; Ladder: Quick-Load 100 bp. (1E) Inverted PCR is used to create linear probe precursors. Gel electrophoresis results confirm the product of inverse PCR. Lanes: 1: Inverted PCR with 200 bp Long Adapter; 2: Inverted PCR with 400 bp Long Adapter; Ladder: Quick-Load 100 bp. A 125 bp pre-LASSO probe was used with either a 220 bp adapter or a 440 bp adapter in the example shown. The pre-LASSO probe is converted to the final LASSO probe by removing the primer annealing sites (e.g., using a combination of a type IIS restriction enzyme and UNG glycosylase) and removing the complementary strand by digestion with exonuclease. Please see “Inverted PCR” in the “LASSO probe assembly” section of the EXAMPLES section below for details.

FIGS. 2A-F. Single ORF target capture with LASSO probes. (2A) Exemplary schematic of single target capture, purification, and amplification. (2B) Post capture PCR of circles obtained from the capture of 620 bp, 1 kb, 2 kb, 4 kb target sequences within the M13Mp18 ssDNA genome using 4 different pre-LASSO probes assembled with a 445 bp adapter. (2C) Post capture PCR of circles obtained from the capture of 620 bp and 1 kb sequences using as template ssDNA M13Mp18, dsDNA M13Mp18 amplicon alone, or dsDNA M13Mp18 amplicon in a background of 10 pM sheared E. coli K12 genomic DNA. (2D) Post capture PCR of circles obtained by capturing a 1,038 bp target sequence within the M13Mp18 dsDNA (˜500 fM) in presence of a equimolar (˜500 fM) background of total genomic DNA of E. coli, using serial dilution of a LASSO probes. Negative controls contain sheared gDNA but no target. (2E) Post capture PCR of circles obtained from the capture of Kanamycin resistance determinant (KanR2) from total DNA (gDNA) or plasmid DNA (pDNA). Negative control for capture was total genomic DNA extracted from an E. coli clone without vector. (2F) Kanamicin resistant E. coli transformant colonies obtained by cloning the post capture PCR of KanR2 into a pET21 expression vector and transformation of BL21 Kanamycin susceptible competent E. coli cells by electroporation. LASSO cloning of the KanR2 gene can thus be used to confer functional resistance to kanamycin.

FIGS. 3A-H. Multiplex capture, sequencing, and cloning of an E. coli ORF library with LASSO probes. (3A) Workflow of an ORFeome capture process using a LASSO probe library. Target sequences are evaluated from metagenomic data with an algorithm used to define criteria for each LASSO probe. A DNA microarray is used to synthesize a pool of oligonucleotides in high density that represents a library of pre-LASSO probes. The pre-LASSO probe pool was converted in a mature LASSO probe pool through a series of reactions in a pooled format. LASSO probes were then hybridized with total genomic DNA of E. coli K12, targeting >3000 ORFs in a single reaction volume. Circles containing ORFs were PCR amplified using primers that hybridize to the conserved adapter region on each LASSO probe. (3B) Post capture PCR of circles obtained from the capture of 3,164 ORFs of E. coli K12 performed by using the LASSO probe library assembled with a 242 bp adapter. The inset is a histogram denoting the target size distribution of the targeted ORFs split into bin size of 40 bp. Short ORFs were used as untargeted internal controls. (3C) Sequencing of the ORF library after LASSO capture using MiSeq. Shown is percentage of on-target and off-target reads of ORFs at a cutoff of 20 reads. (3D) Scatter plot: average coverage per kilobase for each targeted ORF, untargeted ORF and intragenic regions. (3E) ROC analysis; (3F) Positions of captured reads mapped across the normalized, targeted ORFs. Only ORFs having between 100 and 300 reads were included in the graph. (3G) Targeted ORF average coverage as a function of the length of the ORF. (3H) Sanger Sequencing Analysis of a random E. coli clone obtained from the capture library (ORF: NP_414738.1). The chromatogram shows a chimeric sequence at the junctions of the ORF with an adjacent sequence of the LASSO probe as expected. The top inset shows a representative read of the start of an ORF that contains the longer adapter sequence, the ligation arm of the LASSO probe, and the start codon of an ORF. The bottom inset shows a representative read of the end of the selected ORF that contains the fusion site sequence, the extension arm of the LASSO probe, and the stop codon of the selected ORF.

FIGS. 4A-B. Ineffectiveness of Conventional MIPs to Capture Long DNA Fragments. (4A) Amplification of circle derived from the capture of a 100 bp, 400 bp and 980 bp target sequences obtained by using conventional molecular inversion probes (MIPs). The capture was performed by using three ˜120 bp MIPs. After the capture, the circles were PCR amplified using primers that annealed on the backbone sequence. The details of the capture are in the Material and Methods section below. As shown in lane 1, a 100 bp target was captured since there was a DNA band correspondent to the expected amplicon size (170 bp) resulting from the capture of a 100 bp target. A second band at 370 bp was because the polymerization reaction extended around the circle twice. No bands were visible for the 400 bp and 980 bp target sequences (lanes 2 and 3) denoting a failure of conventional MIPs to capture longer fragments. (4B) A proposed model for unsuccessful target capture. A MIP initially hybridized with a longer target is shown on the left. On the right, the complex “unzips” at the ligation arm from the hybridization site due to the stiffness of nascent dsDNA.

FIGS. 5A-B. Optimization of fusion PCR step of single LASSO probe synthesis. (5A) Different amplification and extension conditions of the fusion reaction were tested. Lane 1: Long Adapter (242 bp). Lane 2: Fusion PCR of a pre-LASSO probe (150 bp) with a Long Adapter (242 bp) by direct PCR. Lane 3: Fusion PCR of a pre-LASSO probe (150 bp) with a Long Adapter (242 bp) obtained performing a “fusion by extension” step prior the PCR amplification. The “fusion by extension” involved subjecting the pre-LASSO probe and the Long Adapter to 10 PCR extension cycles (denaturation, annealing and extension) without the primers in the PCR master mix. After the extension, the primers were added in solution and PCR amplification performed for 30 cycles. (5B) Testing different concentrations of pre-LASSO probe (150 bp) and Long Adapters (242 bp, 442 bp) in fusion PCR. As shown in lanes 2, 3, 4; lanes 6, 7, 8 the expected fusion products were obtained by using all three lengths Long Adapters with no visible differences in yield and specificity.

FIG. 6. Optimization of circularization by ligation of fusion PCR products. Two different length fusion PCR products of approximately 370 bp and 570 bp that were obtained from a 150 bp pre-LASSO probe with Long Adapters of 242 bp and 442 bp respectively. Fusion products (1 μg) with sticky ends (EcoRI digested) were diluted to 20 ng/μl and 0.2 ng/μl in 1×T4 DNA Ligase buffer and T4 ligated. After ligation, linear DNA was digested with exonucleases. DNA circles were column-purified, and run in a gel. The reactions were performed by using 20 ng/μl of fusion PCR products, there were DNA circles composed by a single fusion product together with DNA circle composed by concatemers (Lane 1 and 2). The circular nature of the DNA present in the bands was confirmed by the ligase negative controls where all DNA was completely digested by the exonucleases as expected (Lanes 3 and 4). No circular concatemers were visible in the gel when ligation was performed at 0.2 ng/μl (Lane 5 and 6).

FIG. 7. Optimization of Gap Filling mix composition for single target capture using LASSO probes. The aim of this experiment was to compare different DNA polymerases and thermostable DNA ligases gap filling mix formulations in capturing a 100 bp target. Capture was performed by using a LASSO probe that was obtained fusing a 150 bp pre-LASSO probe (pre-LASSO probe 100 bp) and a 242 bp Long Adapter as described in Material and Methods. As shown in Lane 2, the best yield of capture was obtained by using DNA polymerase Omi Klentaq (Enzymatics) in combination with Ampligase DNA Ligase (Epicenter). In the final capture volume the concentration of polymerase was 0.04 U/μl, the final concentration for DNA ligase was 0.02 U/μl, and 100 μM for dNTPs.

FIGS. 8A-B. Estimation of the percentage of functional captured KanR2 ORFs. A pET-21(+) expression vector (ampicillin resistance for selection) was linearized by PCR using tailed-primers with tails identical to the sequence of the primers we used in post capture PCR amplification. Post capture PCR of KanR2 was cloned in pET-21(+) via Gibson Assembly. Transformation of BL21 kanamycin susceptible BL21 E. coli cells was performed by electroporation. (8A) 104 E. coli transformant colonies were replica plated in ampicillin (100n/ml) selection agar plates and ampicillin (100 μg/ml) plus kanamycin (50 μg/ml) selection agar plates. 66 colonies were ampicillin and kanamicin resistant while 38 were ampicillin resistant and kanamycin susceptible. (8B) Colony PCR of the 38 colonies to evaluate the presence of KanR2. Only 4 clones (Lanes 10, 15, 18, 34) contained the KanR2 inserts. Therefore the 34 empty clones were not considered in the estimation of the percentage of functional clones. In total 66 clones were kanamycin resistant, out of the 70 clones that contained the insert. 94% of the captured KanR2 ORFs were therefore functional.

FIGS. 9A-C. Optimization of different parameters for ORFeome capture. (9A) The gap filling mix produced a post capture band pattern that was in agreement with the expected ORF size distribution (Lane 2 and histogram). The gap filling mix formulation developed by Carlson et al. was less suitable for the present method since it produced only faint bands (Lane 1). (9B) Different post capture PCR performed by testing Omni Klentaq (Enzymatics) or ExTaq Polymerase (TaKaRA) at different dNTPs concentrations in the gap filling mix. The best band pattern was obtained by using Omni Klentaq (0.042 U/μl in the final capture volume) with dNTPs 10 μM (in final capture volume). (9C) Captures performed by testing different temperatures for hybridization and capture. The best patterns were obtained when both hybridization and gap filling were performed at 65° C.

FIGS. 10A-B. Fragmentation and Adapter-Ligation of ORF library for MiSeq analysis. Electrophoresis at the Bioanalyzer of a ORF obtained by capturing of 3164 ORFs using a LASSO library long adapter 242 bp.

FIGS. 11A-B. Effect of GC content and melting temperature of individual LASSO probes on ORF target capture.

### DETAILED DESCRIPTION

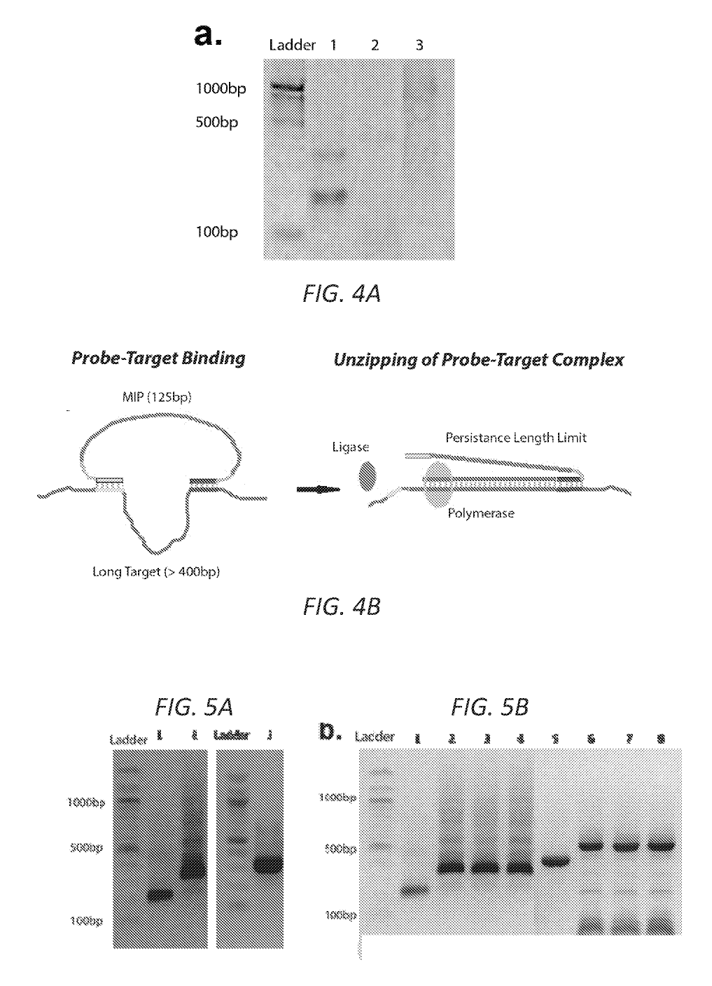

Molecular inversion probes (MIPs) have emerged as an important approach for target DNA sequence enrichment. MIPs hybridize to nearly adjacent DNA sequences, such that the intervening target can be captured by a gap filling and ligation reaction (Nilsson et al., Science 265, 2085-2088 (1994); Landegren et al., J Mol Recognit 17, 194-197 (2004)). However, the efficiency of this reaction drops off dramatically at a target size of ˜200 bp, due to the persistence length (“stiffness”) of double stranded DNA (FIGS. 4A-B). This constraint has prevented its use for the capture of larger fragments, and for the cloning of open reading frames (ORFs) that encode full-length proteins or large protein domains. In an attempt to address this target size limitation, increasing the length of the MIP linker backbone has been shown to permit capture of somewhat longer targets (up to ˜400 bp) (Krishnakumar et al., Proc Natl Acad Sci USA 105, 9296-9301 (2008); Shen et al., Genome Med 5, 50 (2013); Shen et al., Proc Natl Acad Sci USA 108, 6549-6554 (2011)). However, the method used to construct these probes required a separate PCR reaction for each individual probe, thus severely limiting its scalability.

To date, no comprehensive approach to clone the full-length sequence of ORFs from an entire genome sequence (an ORFeome) in a single pooled collection has been described. Present DNA synthesis technologies can make several thousand of different DNA oligonucleotides at the same time on solid surface to be released as a pool (releasable high density DNA microarrays) (Baker, Nature Methods 8, 457-460 (2011)). However, the maximum DNA length achievable by this pooled method is less than 200 nucleotides, which is not long enough for a gene. Currently, methods to produce an ORFeome use the following steps:

1. A pair of primers is designed and synthesized for every single ORF of the organism.

2. Each ORF is amplified by PCR in a separate reaction tube.

3. The PCR product obtained is individually cloned into E. coli. The E. coli clone collection containing ORFs represent the ORFeome.

These three steps need to be repeated for every ORF of the genome, making ORFeome production a long, tedious, and costly process. Multiplex PCR (where multiple primers are added to the same PCR reaction) can simultaneously amplify a few different genes with improvement in time and cost (Caliendo et al., Clin Infect Dis. 52(suppl 4):5326-5330 (2011); Elnifro et al., Clin Microbiol Rev. 2000 October; 13(4):559-70 (2000)). Yet, multiplex PCR cannot be used to amplify a large number of ORFs because of many non-specificity issues. The simultaneous presence of thousands of different primers will inevitably generate preferential target amplification and non-specific byproducts, including primer dimer and mis-priming artifacts (Porreca et al. Nat Methods. 4(11):931-6 (2007); Chou et al., J. Clin Microbiol. 30(9):2307-10 (1992)).

One of the major limitations of studying the functionality of a large pool of bacterial genes is that traditional technologies of manipulating genes are too cumbersome and inefficient when one is dealing with more than a few genes at a time. Entire libraries composed of all protein-encoding open reading frames (ORFs) cloned into highly flexible vectors is critical to rapidly take full advantage of the information found in any genome sequence. The first generation of a proteome in a single phage library at one time constitutes an effective gateway from whole genome sequencing efforts to downstream ‘omics’ applications such as the massive parallel screening.

LASSO

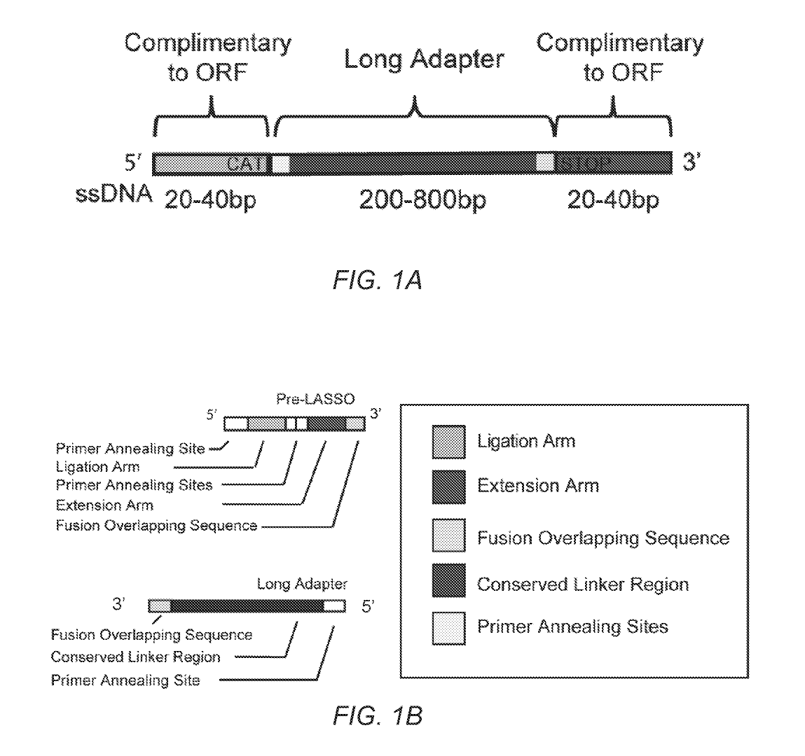

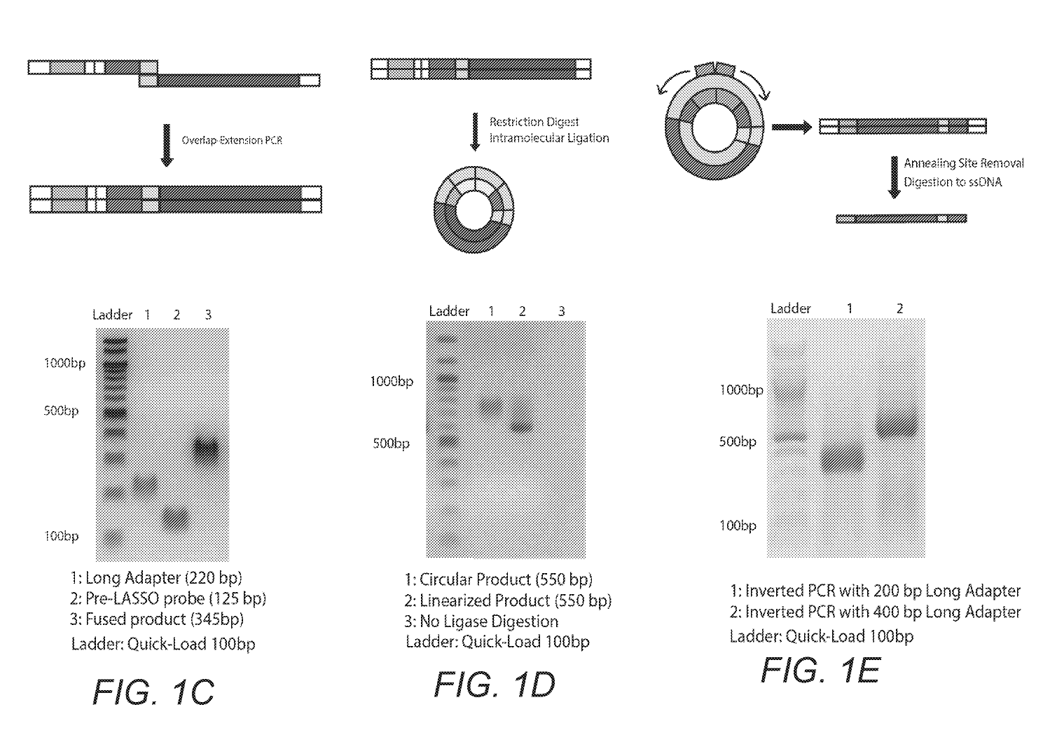

Here, we report the construction and use of Long Adapter Single Strand Oligonucleotide (LASSO) probe libraries (FIG. 1A), which enable the capture of kilobase-sized fragments in a massively multiplexed reaction for downstream sequencing or expression. The methodology presented herein was developed specifically for the assembly of LASSO probes from a complex pool of shorter, synthetic oligonucleotides, which can be readily obtained using programmable DNA microarray synthesis technology (Kosuri and Church, Nat Methods 11, 499-507 (2014)).

The pre-LASSO probe library described herein includes short oligos that are designed to bind a number of target sequences; computer-implemented methods can be used to design the sequences before synthesis. Typically, the library is generated using parallel synthesis to create a pool of probes. This avoids the need to create each probe one by one. Presently synthetic methods allow the generation of synthetic oligos of up to 200 nt, though results are less optimal for oligos over 150-160 nt. The pre-LASSO probes include primer binding sites for inverted PCR sequences which allow the opening of the circular template, after which the sense strand is removed and the complementary strand is used.

The sequences for the primer annealing sites, which are typically 20-50 bp, should not be present in the target genome, and should have no tertiary structure. The sites can also preferably include one or more restriction enzyme recognition sites.

The pre-LASSO probes also include “fusion overlapping sequences” for use in fusing the probes to the Long Adapters; the one exemplified herein was 23 bp, but they can be 15-50 bp, or longer. In some embodiments, all of the pre-lasso probes in the pool have the same fusion overlapping sequences, which are complementary to the fusion overlapping sequences in the Long Adapters.

Alternatively, two (or more) different fusion overlapping sequences can be used (with matching fusion overlapping sequences on different Long Adapters), to provide the option of amplify a sub-pool of the mature library based on a different adapter sequence.

The Long Adapter sequences are non-specific with regard to the target genome and can contain, e.g., one or more restriction sites that would allow digestion after capture and amplification, or a binding site for a protected (e.g., PNA) oligo around priming sites to stop the polymerase and minimize enrichment of particular species or of the adapter probe. This would make for more uniform library. In these embodiments, the methods can include adding a PNA that binds to a region of the Long Adapter after capture; annealing of the PNA creates a very stable DNA/PNA complex with a high melting temperature to stop polymerase processing.

The methods described herein can be used to create libraries of targeted sequences bound with lasso probes. These libraries will generally include the targeted sequences, with some portion of the LASSO probe at one or both ends. The portion of the LASSO probe remaining on the targeted sequence can include, e.g., a barcoding or sequencing primer binding region to allow downstream processing such as sequencing, or restriction sites to facilitate cloning, expression,

LASSO probe-based massively parallel sequence capture promises to become an essential technique for biologists. As the read length of high throughput sequencing technologies continues to increase, there in an unmet need to match the size and scale of corresponding capture fragments. In addition, the ability to rapidly and inexpensively clone large libraries of protein-coding sequences will find many applications in biomedical research and drug development. Here we have demonstrated that LASSO probes can be used to clone thousands of kilobase-sized fragments of DNA (over 3 megabases in total) from a prokaryotic genome. These targeted ORFs included their native start and stop codons, and maintained their intended reading frames. The resulting library of full length ORFs can thus be expressed from standard vectors for subsequent selection or functional characterization. For organisms that splice their mRNA, LASSO probes can also in principle be designed to target cDNA, rather than gDNA, libraries. By design, libraries of protein domains (e.g., extracellular, catalytic, DNA binding, etc.) can be specifically targeted for functional analysis or screening. It may also be possible to clone expressed ORFeomes from tissues or cells using a single, genome-wide LASSO probe set. As the catalog of sequenced genomes and metagenomes continues to grow exponentially, methods to query the functional role of gene products will become increasingly important. Beyond expression cloning, the construction of large-fragment DNA libraries is likely to find many additional applications, especially as deep sequencing technologies evolve and their associated read lengths continue to increase.

Also provided herein are kits for use in the methods described herein. In exemplary embodiments, the kits can include one or more, e.g., all, of the following:

Vial 1: LASSO probes
LASSO Probes

Vial 2: Capture Buffer 10×
Capture Buffer 10×

Vial 3: LASSO Capture Gap Filling Mix
DNA Polymerase
Thermo stable DNA Ligase
dNTPs

Vial 4: Linear DNA digestion solution
Exonuclease I
Exonuclease III
Lambda Exonuclease

Vial 5: Post Capture PCR master mix with primers
DNA polymerase
dNTPs
Primers for Post Capture PCR

An exemplary protocol for the use of such kits is as follows.

1. Prepare DNA template containing targets in Capture Buffer 1× (Vial 1)

2. Add LASSO probes (Vial 2)

3. Hybridize (50-70° C.) for 30′ to more h

4. Add LASSO Capture Gap Filling Mix (Vial 3)

5. Capture the targets (50-70° C.) for 30′ to more h

6. Add Linear DNA Digestion Solution (Vial 4) to digest linear DNA (Template DNA and unreacted LASSO probes)

7. Use one aliquot from 6 and perform the Post Capture PCR using PCR Master mix with Primers provided in Vial 5

8. Post Capture PCR product can be subsequently used for NGS sequencing or Cloning purposes depending on the application.

The Post-Capture PCR products (Step 8) can be used, e.g., with commercial kits to prepare ILLLUMINA libraries or to clone in expression vectors. These libraries (ready-for-sequencing or ready-for-transfection) can be made as specific kits optimized for a number of applications.

### EXAMPLES

The invention is further described in the following examples, which do not limit the scope of the invention described in the claims.

Materials and Methods

The following materials and methods were used in the examples set forth below.

MIP Capture Experiments

MIP capture experiments were performed by using as template a 998 bp DNA fragment of the 16SrDNA of E. coli K12 obtained by PCR using the forward primer CCAGCAGCCGCGGTAATACG (16sRDANAF; SEQ ID NO:1) and the revere primer TACGGTTACCTTGTTACGACTTC (16sRDNAR; SEQ ID NO:2). MIP were 5′P ssDNA oligonucleotide of approximately 120 bp obtained from CCIB (Massachusset General Hospital). Three MIPs were designed in order to capture 100 bp, 400 bp and 980 bp DNA fragments within the template DNA. DNA sequence of the three MIPs were:

5′ctccaagtcgacatcgtttacgGTCTCTGCTGCTTCAGCTTCCCAGTC
GTGGTAGTACATCCATCGTGGTACATACGAGCGATATCCGACGGTAGTGT
TACccccgtcaattcatttgagttt 3′ (MIP100; SEQ ID NO:
3).
5′ctggaattctacccccctctacGTCTCTGCTGCTTCAGCTTCCCAGTC
GTGGTAGTACATCCATCGTGGTACATACGAGCGATATCCGACGGTAGTGT
ACcacaacacgagctgacg-3′ (MIP400; SEQ ID NO: 4)
5′ccgtattaccgcggctgctgGTCTCTGCTGCTTCAGCTTCCCAGTCGT
GGTAGTACATCCATCGTGGTACATACGAGCGATATCCGACGGTAGTGTAC
CCCTACggttaccttgttacgacttc-3′ (MIP 980; SEQ ID
NO: 5)

Lower case sequence indicates the ligation (5′) and extension arms. The hybridization was performed in 15 μl of 1× Ampligase DNA Ligase buffer (Epicentre) containing aproxymately 0.03 pmol of DNA template and 0.01 pmol of MIP. The solution was denatured for 5 min at 95° C., In a PCR thermocycler (Eppendorf Mastercycler), dropped to 60° C., and then let to hybridize for 30 min. The thermocycler program was stopped at 60° C. and 2 μl of gap filling mix were added into the hybridization solution maintaining reaction tube at 60° C. in the thermocycler. The thermocycler program was restarted and the capture was performed for 30 min at 60° C. After capture, the DNA samples were denatured for 3 min at 95° C., dropped to 37° C. and immediately added 2 μl digestion solution. Digestion was performed for 1 h at 37° C. followed by 20 min at 80° C. The gap filling mix composition for a 10 μl volume was: Taq DNA Polymerase (NEB) 2 U, Ampligase DNA Ligase (5 U) dNTPs 200 μM 1× Ampligase DNA ligase Buffer. The digestion solution (volume of 20 μl) was: 10 μl of nuclease free water, 5 μl of Exonuclease I (20 units/μl) and 5 μl of Exonuclease III (100 units/μl) (both from NEB). Post Capture PCR was performed by using 1 μl of the capture reaction containing DNA circles in 25 μl of PCR master mix composed of 0.2 μl Taq DNA Polymerase (NEB) of dNTPs 200 μM, and 0.4 μM of forward primer ATCCGACGGTAGTGTAC (PADperF; SEQ ID NO:6) and reverse primer AGCTGAAGCAGCAGAGA (PADperR; SEQ ID NO:7) that anneal in the conserved backbone of the MIPs.

Pre-Lasso Probes and Long Adapter

Pre-Lasso probe were obtained as double-stranded DNA oligonucleotides

(IDT GBlocks) or as pools of single stranded DNA oligonucleotides derived from programmable DNA microarray (Custom Array inc.). The pre-LASSO probes were approximately 160 bp long and had this design: 3′-GAGTATTACCGCGGCGAATTC, Ligation arm (variable; SEQ ID NO:8), AACACTTCTTGCGGCGATGGTTCCTGGCTCTTCGATC, extension arm (variable; SEQ ID NO:9), AGAGAAGTCCTAGCACGGTAACC-5′(SEQ ID NO:10).

The ORFs of the E. coli K12 genome that are longer than 400 nucleotides were targeted with ligation and extension arms positioned at the beginning and end of the sequences respectively and extended until the desired melting temperature was reached. Specifically, the algorithm first selected the ORF′ leading and trailing 32-mer sequences for the two arms, checking whether the last nucleotide of the arm was a cytosine or a guanine and that the melting temperature for the ligation and extension arms were between 65° C. and 85° C. and 55° C. and 80° C. respectively. If at least one of these conditions were not satisfied, the algorithm increased the length of the arms by one nucleotide and re-tested the conditions until they are satisfied or the end of the ORF is reached. Since an EcoR1 digestion step was used to assemble the LASSO probes, the algorithm discarded the design of pre-LASSO probes where an EcoR1 restriction site was present in the ligation or extension arm.

The Long Adapters (242 bp and 442 bp) were obtained by PCR performed by using tailed primers and as template the plasmid plasmid pCDH-CMV-MCS-EF1-Puro (System Bioscience). The forward primer used for PCR was agagaagtcctagcacggtaaccTCCGAGGATGTCATCAAAGAG (FusionBlaF; SEQ ID NO:11) and was the same for Long Adapter 242 bp and 442 bp), the underlined part represent the tailed region that is identical to the 3′ conserved region of the pre-LASSO probe (above). The reverse primers were aagctggaattcGCTTCCGTACTGGAACTGAGGGC (RFP200EcoR1 for Long Adapter 242 bp; SEQ ID NO:12) and aagctggaattcATGACAGGGCCATCGGAGGGG (RFP400EcoR1 for Long Adapter 442 bp; SEQ ID NO:13). The lower case sequences is the tailed region that contains an EcoRI restriction site. PCR reaction was performed In 25 μl of 1× Klentaq Mutant Buffer containing 0.2 μl of Omni Klentaq LA (DNA Polymerase Technology), 0.4 μM of each primer, dNTPs 200 μM and 10 ng of pCDH-CMV-MCS-EF1-Puro plasmids. The PCR program was 5 min at 95° C.; thirty cycles of 15 sec at 95° C., 20 sec at 55° C., and 40 sec at 72° C.; and 5 min at 72° C. The PCR products was loaded in an 1% agarose gel and DNA band correspondent to the expected size of the Long Adapters were cut and purified from the gel using Wizard SV Gel and PCR Clean-Up System (Promega, USA). The sequences of the 242 bp and 442 Long adapters were:

(SEQ ID NO: 14)
5′agagaagtcctagcacggtaaccTCCGAGGATGTCATCAAAGAGTTTA
AAGAGTTTATGAGATTTAAGGTCAAGATGGAGGGAAGCGTCAACGGACAC
GAGTTCGAGATTGAGGGAGAAGGAGAAGGCCGGCCTTACGAGGGCACACA
AACCGCTAAGCTCAAGGTCACAAAAGGAGGACCCCTCCCCTTCTCCTGGG
ATATTCTGAGCCCTCAGTTCCAGTACGGAAGCgaattccagctt-3′
(SEQ ID NO: 15)
5′agagaagtcctagcacggtaaccTCCGAGGATGTCATCAAAGAGTTTA
AAGAGTTTATGAGATTTAAGGTCAAGATGGAGGGAAGCGTCAACGGACAC
GAGTTCGAGATTGAGGGAGAAGGAGAAGGCCGGCCTTACGAGGGCACACA
AACCGCTAAGCTCAAGGTCACAAAAGGAGGACCCCTCCCCTTCTCCTGGG
ATATTCTGAGCCCTCAGTTCCAGTACGGAAGCAAAGCCTATGTTAAACAC
CCTGCCGACATCCCTGACTATCTGAAGCTCTCCTTCCCTGAAGGCTTCAA
GTGGGAGAGATTCATGAACTTCGAGGACGGAGGCGTGGTGACAGTCACAC
AAGATAGCACCCTCCAGGACGGAGAGTTTATTTATAAGGTGAAACTCAGA
GGAACCAACTTCCCCTCCGATGGCCCTGTCATgaattccagctt

Lower case sequences represent the tails of the primers used for PCR.

LASSO Probe Assembly

Fusion PCR:

The fusion PCR reactions contained: 19 μl of water, 2.5 μl of Klentaq Mutant Buffer 10×, 0.6 μl of dNTPs 10 mM, 0.2 μl of Omni Klentaq LA (DNA Polymerase Technology), 1 μl of water solution containing ˜20 ng of pre-Lasso Probe (whether or not it was a single dsDNA pre-Lasso probe or a pool of ssDNA pre-Lasso probes), 1 μl of water solution ˜20 ng of Long Adapter. The solution was denatured 4 min at 95° C. and subjected to 10 thermal cycles as follow; 15 sec at 95° C., 20 sec at 50° C., 40 sec at 72° C. After the 10 cycles the PCR was stopped and 2 μl of water solution of 5 μM fusion primers (1 μl of 10 μM Fusion Primers forward BLAF and 1 μl of 10 μM Fusion Primer reverse (RFPR200EcoR1 or RFPR400EcoR1, depending on which long adapter is being fused) was added in solution. The PCR tubes were subsequently subject to 30 more cycles: 15 sec at 95° C., 20 sec at 50° C., 40 sec at 72° C.

The sequence of the primer was GAGTATTACCGCGGCGAATTC (BLAF; SEQ ID NO:16) and is identical to the 5′ conserved region of the pre-LASSO probe. The RFPR200EcoR1 and RFPR400EcoR1 are the same that were used to obtain the Long Adapter.

Fusion PCR products (approximately 26 μl for each reaction) were split in two 13 μl aliquots, added the loading dye, and subjected to agarose gel electrophoresis using a 1.1% agarose gel. DNA bands correspondent to the expected sizes of the fusion PCR products were recovered from the gel by cutting with a scalpel. DNA was purified by using QIAquick Gel Extraction Kit (Quiagen) or Wizard SV Gel and PCR Clean-Up System (Promega) and eluted in 50 μl of water final volume.

Self-Circularization:

The approximately 45 μl solution containing gel purified fusion PCR product as described above were digested by adding 5 μl of EcoRI 10× buffer and 1 μl (20 units/μl) of EcoRI restriction enzyme (NEB) for 1 h at 37° C. followed by 10′ at 80° C. The digested DNA was purified using AmpPure beads (1.4× and washed with ETOH 70%) and eluted in 40 μl of water. Self-circularization was performed in a total volume of 50 μl of 1×T4 Ligase Buffer (NEB) containing approximately 5 ng of EcoRI digested fusion PCR product (0.1 ng/μl) and 1 μl of T4 DNA ligase (400 units), DNA ligase was added last. The reaction was performed in a thermocycler (Eppendorf Mastercycler) for 30 min at 25° C. followed by 10 min at 65° C. Non Self-circularized DNA was digested by adding 2 μl of solution containing 1 μl of Lambda Exonuclease (5 U/μl) and 1 μl of Exonuclease I (20 U/μl) (both purchased from NEB) directly into the PCR tube containing the self-circularized DNA. Digestion proceeded at 37° C. for 30 min followed by 20 min at 80° C.

Inverted PCR:

Inverted PCR was performed in a 25 μl total volume containing 10 μl of the Self-circularized DNA as described above, 2.5 μl of Klentaq Mutant Buffer 10×, 0.2 μl of Omni Klentaq LA (DNA Polymerase Technology), 0.6 μl of dNTPs (NEB), 1 μl of 0.4 μM reverse primer A*T*C*GCCGCAAGAAGTGTU (ThiolR; SEQ ID NO:17), 1μ of 0.4 μM forward primer GGTTCCTGGCTCTTCGATC (SapIF; SEQ ID NO:18) and 10 μl of water. Both SapI and ThiolR anneal with opposite orientations in the conserved central section of the pre-LASSO probe (AACACTTCTTGCGGCGATGGTTCCTGGCTCTTCGATC; SEQ ID NO:18). The SapIF primer contains a SapI restriction site, the * indicates phosphorothioate bonds, U indicate a deoxyuracil moiety. The PCR thermal profile was 4 min at 95° C.; thirty cycles of 10 sec at 95° C., 20 sec at 55° C., 40 sec at 72° C.; 4 min at 72° C.

The inverted PCR product was subsequently purified by using AmpPure beadsbeads (1.4×), washed with ETOH 70%) and eluted with 40 μl of nuclease free water. The concentration of purified inverted PCR product was measured by Nanodrop.

Production of Mature LASSO Probes:

Approximately 1 μg of purified Inverted PCR product were digested by adding 4 μl of CutSmart buffer 10× (NEB) and 1 μl of SapI restriction enzyme (NEB). Digestion was performed at 37° C. for 1 h followed by 20 min at 65° C. After digestion, 1 μl (5 units) of Lambda exonuclease (NEB) was added directly to the SapI digested DNA and for 30 min at 37° C. followed by 10 min at 80° C. for enzyme inactivation. At this point 2 μl (1 unit/0 of USER enzyme (NEB) were added in solution and incubated for 30 min at 37° C. Finally the mature ssDNA form of Lasso Probes were purified using AmpPure beads (1.4× and washed with ETOH 70%) and eluted in 40 μl of water. The final concentration of mature ssDNA LASSO probes was determined by Nanodrop. Typically, starting from 1 μg of purified Inverted PCR product, the yield was approximately 400 ng. DNA templates used in capture experiments: For LASSO probe capture optimization experiments, we used a 7249 bp circular, single-stranded DNA isolated from the M13mp18 phage (NEB) or alternatively the double-stranded, covalently closed, circular form of DNA derived from bacteriophage M13 (NEB).

For capture experiments of E. coli ORFeome, total genomic DNA of the E. coli strain K12 substrain W3110, (Migula) Castellani and Chalmers (ATCC 27325) was extracted from 500 μl of LB broth (Sigma Aldrich) overnight culture using Charge Switch gDNA Mini Bacteria Kit (Life technology). Sheared total genomic DNA of E. coli K12 was obtained by sonicating 1 μg of total DNA in a volume of 200 μl in a 1.5 ml Eppendorf tube on ice by using a Branson sonifier 450 (VWR scientific) at output control 2, duty cycle 50% for 40 sec.

For the capture of the 815 bp long kanamycin resistance gene KanR2 we used total DNA of the E. coli clone n 29664 (Addgene) that contained the pET StrepII TEV LIC cloning vector harboring KanR2 gene.

Hybridization and Capture of E. coli ORFeome:

For the capture of the 3164 E. coli K12 ORFs, the hybridization was performed in 15 μl of 1× Ampligase DNA Ligase buffer (Epicentre) containing: 100 ng of unshared E. coli K12 total genomic DNA and 100 ng of shared E. coli K12 total genomic DNA and 4 ng of LASSO probes pool. In solution there was approximately 0.06 fmol of E. coli chromosomes and 4 amol for individual LASSO probes (˜12 fmol of LASSO probe pool).

Sheared E. coli K12 DNA was obtained by sonicating 1 μg of total genomic in 200 μl total volume in a Eppendorf tube on ice by using a Branson sonifier 450 (VWR scientific) at output control 2, duty cycle 50% for 30 sec.

The solution (15 μl) containing the LASSO probe pool and the E. coli DNA, was denatured for 5 min at 95° C. in a PCR thermocycler (Eppendorf Mastercycler), then incubated at 60° C. for 60 min.

After hybridization 5 μl of freshly prepared gap filling mix were added into the hybridization solution, while maintaining the reaction at 60° C. in the thermocycler. Gap filling and ligation was performed for 30 min at 60° C. After capture, the DNA samples were denatured for 3 min at 95° C., and the temperature reduced to 37° C. 2 μl Linear DNA Digestion Solution was added immediately. Digestion was performed for 1 h at 37° C., followed by 20 min at 80° C.

Gap Filling Mix was prepared fresh for each capture experiments and the composition for 50 μl of gap filling mix was: 2 μl of 1 mM dNTPs, 1 μl of Ampligase DNA Ligase (5 U/μl), 2 μl of OmniKlenTaq LA that was previously diluted 1/10 in 1× Ampligase DNA Ligase Buffer, 5 μl of Ampligase DNA ligase Buffer 10×, 40 μl of DNAase free water. Linear DNA Digestion Solution (volume of 20 μl) was composed by 10 μl of nuclease free water, 5 μl of Exonuclease I (20 units/μl) and 5 μl of Exonuclease III (100 units/μl) (both from NEB).

Hybridization and Capture of Different DNA Targets Using Single LASSO Probes:

The capture of the 620 bp, 1 kb, 2 kb and 4 kb target sequences located in the DNA of the phage M13 were performed with the same gap filling mix composition and the same thermal profile for hybridization and capture used for the LASSO probe pool as described above. We used approximately 0.3 fmol of single LASSO probes, and 4 fmol of M13Mp18 dsDNA or ssDNA. The E. coli k12 total genomic DNA background was 10 pM (500 ng DNA in 15 μl capture volume).

For the LASSO probe sensitivity test, E. coli k12 total genomic DNA background was ˜500 fM (25 ng in 15 μl capture volume). The concentration of M13Mp18 dsDNA was ˜500 fM (0.03 ng in 15 μl). The serial dilution concentration of the LASSO 1 kB probe were 500 pM, 50 pM, 5 pM and 500 fM.

Capture of KanR2 gene was performed by using 20 ng of total genomic DNA of E. coli clone n 29664 (Addgene) 3 fmol of LASSO probe KnaR2 (pre-LASSO KnaR2 assembled with 442 bp Long Adapter). Capture was performed using the same gap filling mix and thermal profile used for the LASSO probe pool. The DNA sequences of single pre-LASSO probes are in Table 1.

TABLE 1
Single Pre-LASSO probes

| | | SEQ |
|---|---|---|
| Oligo | | ID |
| Name | Sequence | NO: |
| Pre- | GAGTATTACCGCGGCGAATTCATGAGCCATATTCAACGGGAAA | 20 |
| LASSO | CGTCTTGCTCTAGGAACACTTCTTGCGGCGATAGAAGGTTCCT | |
| KanR2 | GGCTCTTCGATCGCAGTTTCATTTGATGCTCGATGAGTTTTTC | |
| | TAAAGAGAAGTCCTAGCACGGTAACC | |
| Pre- | GAGTATTACCGCGGCGAATTCCCAACGGCAGCAGCGGATCCGT | 21 |
| LASSO | GAACACTTCTTGCGGCGATAGAAGGTTCCTGGCTCTTCGATCT | |
| 100 | GATTTATGGTCATTCTCGTTTTCAGAGAAGTCCAGCACGGTA | |
| bp | ACC | |
| Pre- | GAGTATTACCGCGGCGAATTCTTGGAGTTTGCTTCCGGTCTGG | 22 |
| LASSO | TTCGCAACACTTCTTGCGGCGATAGAAGGTTCCTGGCTCTTCG | |
| 620 | ATCGATTTGGGTAATGAATATCCGGTTCTTGTCAAGAGAGAAG | |
| bp | TCCTAGCACGGTAACC | |
| Pre- | GAGTATTACCGCGGCGAATTCTTGGAGTTTGCTTCCGGTCTGG | 23 |
| LASSO | TTCGCAACACTTCTTGCGGCGATAGAAGGTTCCTGGCTCTTCG | |
| 1 kb | ATCGCCGTTGCTACCCTCGTTCCGATGCAGAGAAGTCCTAGCA | |
| | CGGTAACC | |
| Pre- | GAGTATTACCGCGGCGAATTCTTGGAGTTTGCTTCCGGTCTGG | 24 |
| LASSO | TTCGCAACACTTCTTGCGGCGATAGAAGGTTCCTGGCTCTTCG | |
| 2 kb | ATCGGCTCTGAGGGTGGCGGTTCTGAGGAGAGAAGTCCTAGCA | |
| | CGGTAACC | |
| Pre- | GAGTATTACCGCGGCGAATTCTTGGAGTTTGCTTCCGGTCTGG | 25 |
| LASSO | TTCGCAACACTTCTTGCGGCGATGGTTCCTGGCTCTTCGATCG | |
| 4 kb | GCGAATCCGTTATTGTTTCTCCCGATGTAAGAGAAGTCCTAGC | |
| | ACGGTAACC | |

Post Capture PCR:

The captured ORFs were amplified using 5 nl of the capture reaction containing DNA circles in 25 nl of PCR master mix composed of 0.3 nl of Omni Klentaq LA (DNA Polymerase Technology), dNTPs 200 μM, and 0.4 μM of primers that annealed on the Long Adapter sequence. Depending on the Long Adapter sequence length (242 bp or 442 bp), the primers for amplification were: CAAACCGCTAAGCTCAAGGTCACAAAAGG (FRPLoopF; SEQ ID NO:26) and CGCTTCCCTCCATCTTGACCTTAAATCTCA (PCR1kbCaptR200; SEQ ID NO:27) for the 242 bp Long Adapter; the primers GTGAAACTCAGAGGAACCAACTTCC (PCR1kbCaptF400; SEQ ID NO:28) and CGCTTCCCTCCATCTTGACCTTAAATCTCA (PCR1kbCaptR200; SEQ ID NO:29) were for the 442 bp Long Adapter.

The PCR thermal profile was 4 min at 95° C.; 30 cycles of 10 sec at 95° C., 20 sec at 55° C., and 2 min at 72° C.

To visualize the amplicons derived from the circles, 6 μl of PCR products were loaded in a 1.1% agarose gel containing ethidium bromide (0.2 μg/ml) and visualized using a UV transilluminator.

Expression Cloning:

PCR amplicons were cloned via Gibson Assembly in the vector pET-21(+) (Novagen) that was previously linearized by PCR using tailed-primers tcctctgagtttcacCGGATCCGCGACCCATTTGC (pET21RGibson; SEQ ID NO:30) and tcaagatggagggaagcgAATTCGAGCTCCGTCGACAA (pET21FGibson; SEQ ID NO:31). Lower case sequences represent the tails of the primers that overlap the sequence of the primers used in post capture PCR (PCR1kbCaptR200, and PCR1kbCaptF400). Gibson Assembly reaction was performed as described by the vendor (NEB). Transformation of BL21 elecrocompetent E. coli cells (Sigma) was performed using a 0.1 cm cuvette (Bio Rad) and a Bio Rad Micro Pulser. E. coli transformed clones were selected with agar plates containing ampicillin (100 μg/ml).

Sanger Sequencing:

Post capture PCR products were cloned into pMiniT(NEB) by using NEB PCR cloning kit and used to transform chemically competent NEB 10-beta E. coli cells (NEB) as described by the vendor. Single colonies of transformed E. coli clones were picked from selective plate containing ampicillin (100 μg/ml). The presence of DNA inserts was determined by using the colony as DNA template for PCR with the primers provided with the kit. PCR product (5 μl) were visualized by agarose gel electrophoresis and purified using AmpPure beads. Sanger sequencing of cloned amplicons was performed by capillary electrophoresis on the 96-well capillary matrix of an ABI3730XL DNA Analyzer.

Illumina Library Construction:

Post capture PCR products (25 μl) were purified using magnetic beads Agencourt AMPure XP system and eluted in 40 μl of water. The DNA concentration was measured at the Nanodrop. Purified Post capture PCR (200 ng DNA) were collected, brought to 50 μl with nuclease free water and sonicated in an eppendorf tube on ice using a Branson sonifier 450 at output control 2, duty cycle 50% for 30 sec.

The sheared DNA was subjected to end repair, 5′ phosphorylation, dA-tailing and Illumina adaptor ligation using the NEBNext Ultra DNA Library Prep Kit for Illumina (NEB) as described by the vendor. PCR enrichment of adaptor ligated DNA was performed using NEBNext Multiplex Oligos (NEB) with index primers. Thermal profile was: 30 sec at 98° C., 8 cycles of 10 sec at 98° C., 75 sec at 63° C., and, 5 min at 72° C. PCR products were finally purified using Agencourt AMPure XP system as described in the NEB protocol. The quality of the Illumina library was verified by checking the size distribution on an Agilent Bioanalyzer using a high sensitivity DNA chip. The concentration of the Illumina library was measured by qPCR using the NEBNext Library Quant Kit for Illumina (NEB). DNA sequencing was performed by using the Illumina MiSeq device with the MiSeq Reagent Kit v3 (Illumina).

Illumina Sequence Processing:

Samples were sequenced using the Illumina MiSeq v3 platform according to the manufacturer's instructions. To improve cluster generation for these low complexity libraries, we spiked in PhiX or whole genomic DNA libraries at 10%-20%. We collected one 250-bp forward read to determine sequence of the ligation arm and STR target locus, one 50-bp reverse read to determine the sequence of the degenerate tag and extension arm, and one 8-bp read to determine the sample index sequence. The MiSeq software sorted by index read to separate pooled libraries. Illumina reads were mapped against the E. coli K12 reference genome sequence using BowTie2 (Langmead and Salzberg, Nat Methods 9, 357-359 (2012)). The resulting alignment was processed with SAMtools (Li et al., Bioinformatics 25, 2078-2079 (2009)) to determine the coverage of each nucleotide position and the average coverage of target ORFs, non-target ORFs and intergenic regions.

Statistical Analysis:

All data are presented in mean±standard error of the mean (SEM), as stated in the figure legends. Statistical significance was assessed using Student's t-test for pair-wise comparison, and 1-way ANOVA for comparison between multiple (>3) conditions; p<0.05 was considered as significant.

### Example 1. Long Adapter Single Stranded Oligonucleotide Probes to Capture and Clone Complex Libraries of Kilobase-Sized DNA Fragments

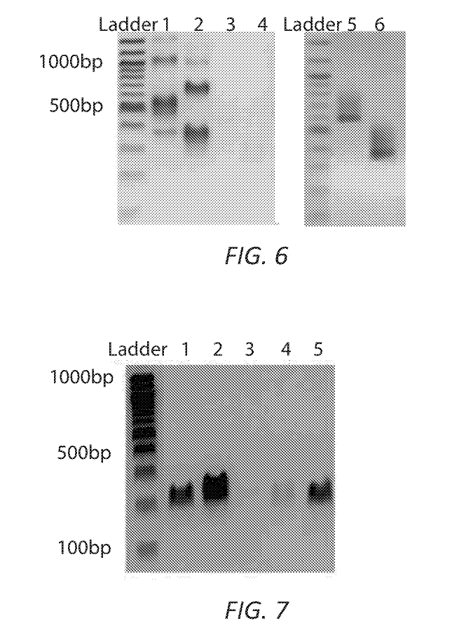

In an exemplary method, LASSO probe construction began with the fusion of a precursor probe (pre-LASSO probe; Table 1), designed to hybridize with sequences that flank the targeted region, and a Long Adapter sequence (FIG. 1B). The fusion of long adaptor and pre-LASSO probe occurred with better specificity if the hybridized complex was extended prior to amplification (FIG. 5A) and was efficient at varying concentrations of adapter and at different pre-LASSO probe lengths (FIG. 5B). The resulting pre-LASSO fusion product was then circularized (FIG. 1D) and subjected to inverse PCR, so that the LASSO annealing arms were made to flank the long adapter sequence (FIGS. 1E and 6). The external primer sites were next removed and the final ssDNA LASSO probe was produced by exonuclease digestion. The final LASSO probe pool was purified and ready to use in massively parallel target sequence capture reactions.

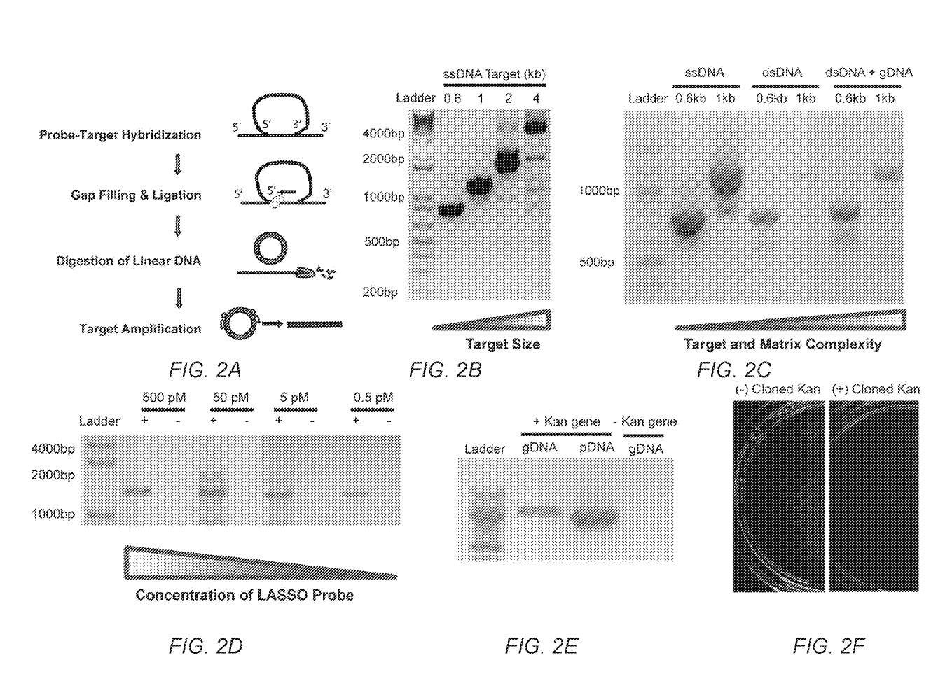

LASSO probes were initially evaluated for their ability to clone long DNA targets, at first by fusing a 150 bp pre-LASSO probe and a 242 bp Long Adapter. The capture reaction involves a multi-step process of annealing, extension, ligation, digestion, and amplification of the probe-target complex (FIG. 2A). Starting with a 100 bp target, we used single target reactions to determine the optimal conditions for gap filling and ligation (FIG. 7). Four LASSO probes (fused with a 442 bp Long Adapter) were designed to capture four different target DNA sequences of approximately 0.6 kb, 1 kb, 2 kb, and 4 kb in size, located within the ssDNA genome of the M13 bacteriophage. All four probes were able to capture their targets with high specificity (FIG. 2B).

We assessed the influence of target DNA strandedness and background matrix complexity. The same concentration of LASSO probe was applied to M13 ssDNA, the corresponding M13 dsDNA produced by PCR, and M13 dsDNA in presence background of sheared E. coli whole genomic DNA. Under these conditions, we observed capture efficiency to decrease using dsDNA as a target, versus ssDNA. Efficiency was recovered, however, when the dsDNA template was first melted within a complex matrix of sheared gDNA (FIG. 2C). This finding is consistent with dsDNA target re-hybridization, which would compete with LASSO probe annealing. Next, a dilution series of a LASSO probe was performed to test the sensitivity of the reaction, and the feasibility of performing massively multiplexed reactions that include thousands of LASSO probes (individually at low concentration) in the same reaction. A 1 kb dsDNA target sequence (500 fM) was spiked into an equimolar background of E. coli gDNA in order to simulate capture of a single copy target gene. We detected captured product even at the lowest dilution of the LASSO probe tested (500 fM) (FIG. 2D). Importantly, “off target” products were not observed when the target sequence was absent from the reaction (which still contained the background gDNA), thus highlighting the specificity of the capture reaction.

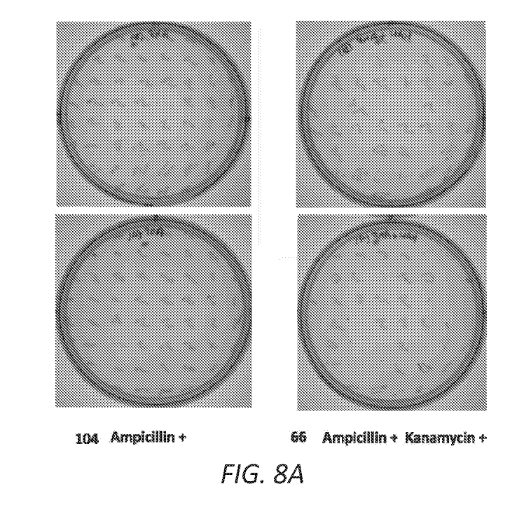

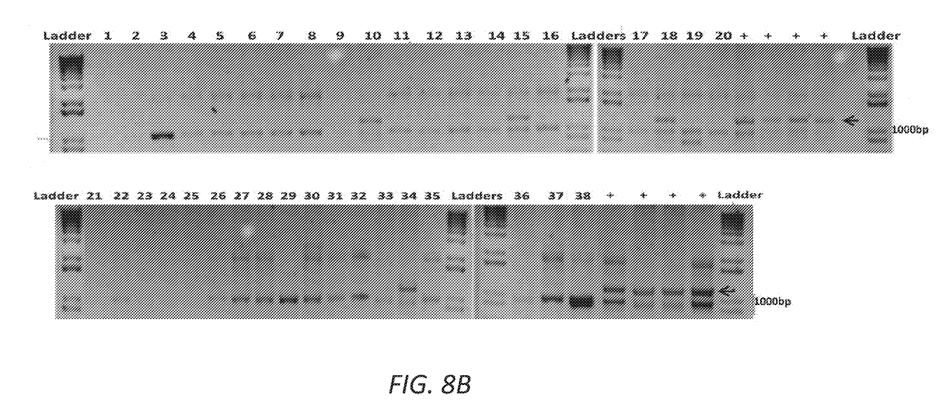

An important application for the capture of long DNA sequences is efficient cloning of ORF libraries for protein expression screening. We therefore assessed the fidelity of LASSO probe-based cloning of the kanamycin resistance gene (KanR2, 815 bp) from a DNA vectors. The KanR2 gene was captured successfully from total gDNA or a plasmid DNA template (FIG. 2E), and cloned via Gibson Assembly into pET-21(+) vector. Dual selection of ampicillin (present in pET-21(+)) and kanamycin demonstrated that 93% of the captured KanR2 genes could be functionally expressed (FIGS. 2F and 8A-B).

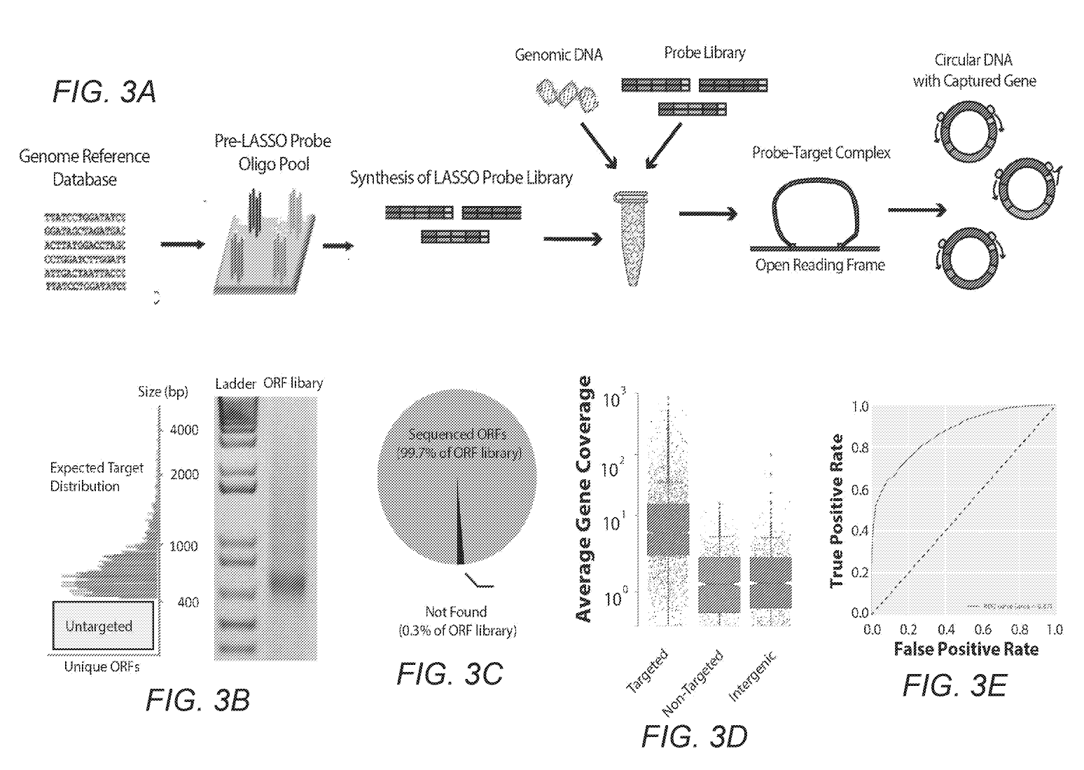

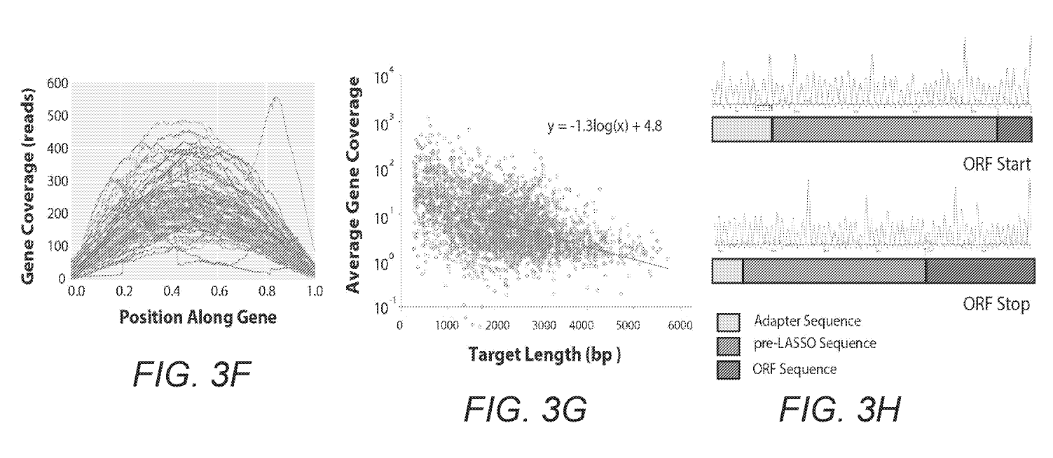

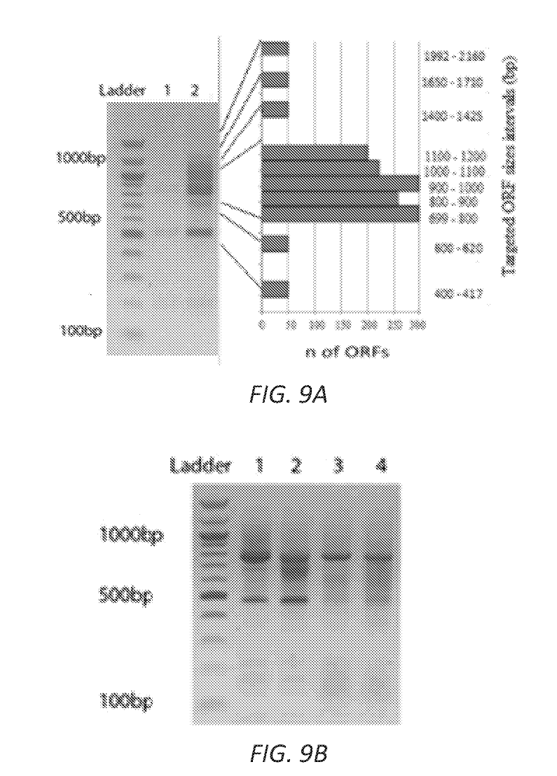

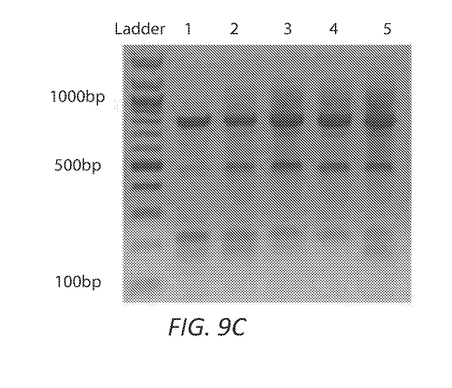

We next assessed the performance of LASSO probes for the massively multiplexed cloning of a library of kilobase-sized ORFs from E. coli genomic DNA (FIG. 3A). ORFeome cloning is a particularly stringent test of multiplexed long sequence capture, since the design of probe sequences is highly constrained by the sequences downstream and upstream of each ORF's start and stop codons, respectively. Using parameters defined by our optimization experiments, we developed a LASSO probe design algorithm, which we used to generate thousands of pre-LASSO probe sequences. Of the 3,999 annotated E. coli K12 (ATCC 27325) ORFs, the algorithm produced 3,664 pre-LASSO probe sequences that satisfied our requirements (˜92% of targets). Adjusting the thresholds for target length, melting temperature, or the length of the ligation/extension arms determines the number of acceptable probes. Of the 3,664 acceptable probes, we removed those corresponding to targets smaller than 400 nt, as a precaution to avoid potentially skewing our capture library during its subsequent PCR amplification. Approximately 20% of the E. coli K12 ORFeome was left untargeted (835 ORFs) and thus served as an internal, negative control for our experiments (FIG. 3B). A programmable DNA microarray was used to synthesize the pool of 3,164×160 bp pre-LASSO probes. These precursor probes were then converted into a mature LASSO probe library (adapter length=242 bp). A series of optimization experiments were performed on library capture conditions using a partial ORFeome (FIGS. 9A-C). In 2015 Omni Kleantaq was discontinued by Enzymatics. We started purchasing the same enzyme from DNA Polymerase Technology, Inc. with the name of Omni Kleantaq LA. Since the title of the enzyme (U/μl) is not indicated, we established the appropriate amount for the gap filling mix. We find that we were able to obtain the same capture results by diluting it before adding it to the gap filling mix as described in Material and Methods. Our gap filling mix is composed of 0.025 U/μl of Ampligase DNA Ligase in final capture volume. Different authors used much higher concentrations of Ampligase DNA Ligase in the final capture volume: Brian J. O'Roak et al. (Science 21, 338 (2012)) 1 U/μl, Carlson et al. (Genome Res 5, 750-761 (2015)) 3 U/μl, Jin Billy Li et al. (Genome Res 19, 1606-15. (2009)) 0.16 U/μl, Peidong Shen (Proc Natl Acad Sci USA. 108, 6549-54 (2011)) 0.25 U/μl. We investigated whether increasing the concentration of the Ampligase DNA Ligase up to 1 U/μl (maintaining Omni Klentaq at 0.042 U/μl and dNTPs 10 μM) could improve the capture efficiency. We noticed no differences in yield or band pattern (data not shown) indicating that 0.025 U/μl of Ampligase DNA Ligase in final capture volume was sufficient for capture.

As shown in FIG. 9A, the gap filling mix produced a post capture band pattern that was in agreement with the expected ORF size distribution (Lane 2 and histogram). The gap filling mix formulation developed by Carlson et al. was less suitable for the present method since it produced only faint bands (Lane 1). FIG. 9B shows different post capture PCR performed by testing Omni Klentaq (Enzymatics) or ExTaq Polymerase (TaKaRA) at different dNTPs concentrations in the gap filling mix. The best band pattern was obtained by using Omni Klentaq (0.042 U/μl in the final capture volume) with dNTPs 10 μM (in final capture volume). FIG. 9C shows captures performed by testing different temperatures for hybridization and capture. The best patterns were obtained when both hybridization and gap filling were performed at 65° C.

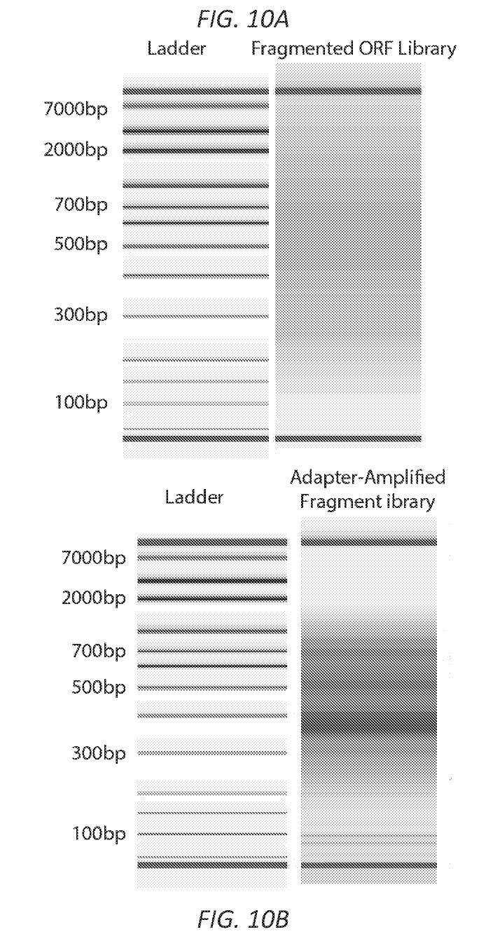

Resulting PCR-amplified ORFs are shown in FIG. 3B, and their apparent size distribution corresponded well with that of the targeted ORFs. The PCR amplicon was sheared (FIGS. 10A-B) and sequenced on an Illumina MiSeq instrument (150 bp paired-end reads). Of the reads that aligned perfectly to the E. coli K12 genome, 99.7% of these mapped onto one of the targeted ORFs with a minimum threshold of 20 reads, whereas the remaining 0.3% mapped to the untargeted 20% of the E. coli K12 ORFeome (FIG. 3C). FIG. 3D illustrates the distribution of read counts per kilobase for each targeted ORF, untargeted ORF and intragenic region. Targeted ORFs were significantly enriched of over non-targeted ORFs and intergenic regions (P=8×10⁻⁷⁸; no significant difference between non-targeted ORFs and intergenic regions) with a high positive predicted value (0.87) as determined by ROC analysis (FIG. 3e). Our data indicate that 89.4% of the cloned library is present within 10-fold abundance of the median. Interestingly, most of the targeted ORFs that were not sequenced at all in our cloned library actually encode mobile genetic elements such as transposases and prophages (Table 2), suggesting their potential absence from the template material.

TABLE 2
Missing Targeted ORFs

| | | Length |
|---|---|---|
| ORF Name | | (bp) |
| 418760.1 | putative DNA-binding transcriptional | 1413 |
| | regulator/putative aminotransferase | |
| 416434.1 | flagellar filament capping protein | 1407 |
| 414801.1 | CP4-6 prophage; putative DNA-binding | 1155 |
| | transcriptional regulator | |
| 415922.1 | IS30 transposase | 1152 |
| 416318.1 | ribonuclease D | 1128 |
| 415279.1 | galactose-1-phosphate uridylyltransferase | 1047 |
| 415189.1 | IS5 transposase and trans-activator | 1017 |
| 415280.3 | UDP-galactose-4-epimerase | 1017 |
| 417456.1 | IS5 transposase and trans-activator | 1017 |
| 416696.1 | IS5 transposase and trans-activator | 1017 |
| 416535.1 | IS5 transposase and trans-activator | 1017 |
| 415847.1 | IS5 transposase and trans-activator | 1017 |
| 417685.1 | IS5 transposase and trans-activator | 1017 |
| 415084.1 | IS5 transposase and trans-activator | 1017 |
| 415288.1 | 6-phosphogluconolactonase | 996 |
| 418715.2 | putative DNA-binding transcriptional | 987 |
| | regulator; KpLE2 phage-like element | |
| 416603.1 | putative kinase | 966 |
| 416065.1 | Qin prophage; putative side tail fiber | 963 |
| | assembly protein | |
| 415289.4 | putative DNA-binding transcriptional regulator | 954 |
| 416029.1 | lsr operon transcriptional repressor | 954 |
| 414857.1 | carbamate kinase-like protein | 951 |
| 026285.1 | uncharacterized protein | 939 |
| 415906.1 | ring 1,2-phenylacetyl-CoA epoxidase subunit | 930 |
| 415920.1 | IS2 transposase TnpB | 906 |
| 417337.1 | IS2 transposase TnpB | 906 |
| 416500.1 | IS2 transposase TnpB | 906 |
| 417517.1 | IS2 transposase TnpB | 906 |
| 414786.4 | CP4-6 prophage; conserved protein | 822 |
| 416835.2 | DUF2544 family putative outer membrane protein | 822 |
| 415039.1 | transcriptional repressor of all and gcl | 816 |
| | operons; glyoxylate-induced | |
| 416430.1 | cystine transporter subunit | 801 |
| 418087.1 | kinase that phosphorylates core heptose | 798 |
| | of lipopolysaccharide | |
| 026280.1 | NADH pyrophosphatase | 774 |
| 415595.1 | flagellar component of cell-proximal portion | 756 |
| | of basal-body rod | |
| 416077.4 | Qin prophage; putative antitermination protein Q | 753 |
| 416427.1 | putative ABC superfamily transporter | 753 |
| | ATP-binding subunit | |
| 415878.1 | Rac prophage; putative DNA replication protein | 747 |
| 416490.1 | UPF0082 family protein | 717 |
| 417123.1 | CP4-57 prophage; putative DNA-binding | 702 |
| | transcriptional regulator | |
| 415754.1 | thymidine kinase/deoxyuridine kinase | 618 |
| 416438.1 | lipoprotein | 414 |
| 417570.1 | DUF1469 family inner membrane protein | 405 |

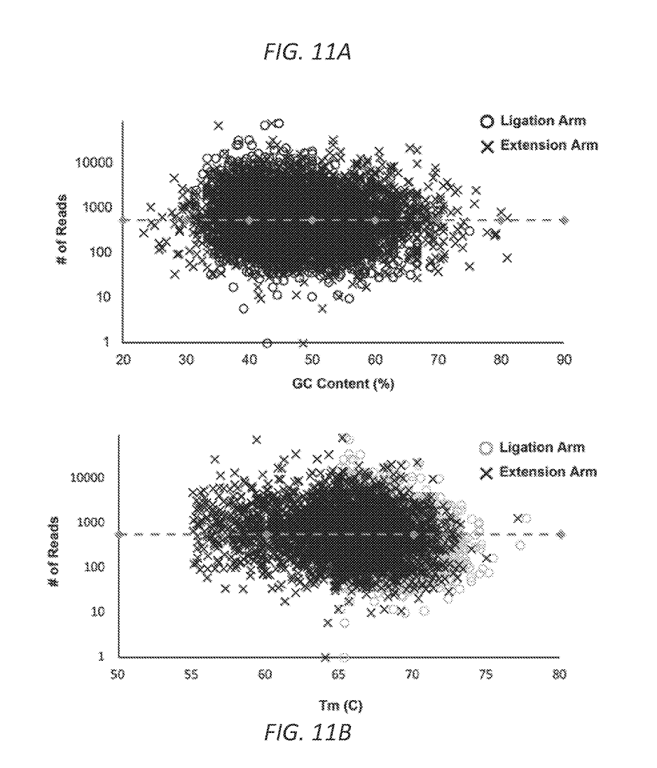

Neither the LASSO probes' GC content nor their melting temperatures were associated with any identifiable skewing of the on-target reads (FIGS. 11A-B). After filtering out adapter-containing sequences, the frequency of mapped sequence reads were plotted according to their normalized position within the corresponding ORF (FIG. 3F). Several randomly selected target ORFs were also examined in this way individually. We observed no enrichment for sequences adjacent to the start or stop codons, suggesting that the vast majority of sequencing reads came from full length ORFs and that internal ORF positions were represented uniformly in our capture library. We observed a correlation between the representation of each ORF and its length. FIG. 3G illustrates that ORF representation within the library declines by 60% at each doubling of its length. This may reflect target length-dependent capture efficiency, post capture PCR bias, or a combination of the two effects.

The integrity of the ORFs was also confirmed by Sanger sequencing of 20 E. coli transformants that were obtained by cloning the capture in a vector for sequencing. An abridged sequence of the start and stop regions of a representative cloned ORF is shown in FIG. 3H. As shown, the sequence contains the long adapter between the primer used for post capture PCR and the ligation arm, the ATG start codon followed by the complete captured ORF, and the sequence of the long adapter between the STOP codon and the primer used for PCR. These data provide unique evidence that the cloned sequence was derived from a LASSO capture given the presence of the adjacent pre-LASSO and adapter sequences.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A Long Adapter Single Stranded Oligonucleotide (LASSO) comprising, from 5′ to 3′:
a ligation arm sequence of 20-40 nucleotides (nt) complementary to a 5′ region of a target sequence;
a Long Adapter sequence of 200 to 2500 nt, comprising a fusion overlapping sequence and optionally one or more restriction enzyme recognition sites;
an extension arm sequence that is 15-80 nt, complementary to a 3′ region of a target sequence,
wherein the ligation arm and extension arm sequences are complementary to 5′ and 3′ regions of a single target sequence and the complementary regions are at least 200-30,000 nts apart on the target sequence, and wherein the Long Adapter sequence is not complementary to the target sequence.

2. The oligonucleotide of claim 1, wherein the target sequence is a coding or noncoding DNA sequence including complete or partial open reading frames, complete or partial intronic DNA regions or other noncoding sequence such as lincRNA or regulatoryRNA.

3. A plurality of the oligonucleotides of claim 1, wherein the plurality includes oligonucleotides with sequences complementary to 10 or more different target sequences.

4. A plurality of pre-LASSO probes, wherein the pre-LASSO probes are 80-200 base pairs (bp) long, comprising (i) a ligation arm sequence of 15-80 bp that is complementary to a 5′ region of a target sequence, (ii) an extension arm sequence of 15-80 bp that is complementary to a 3′ region of a target sequence, wherein the ligation arm and extension arm sequences are complementary to 5′ and 3′ regions of a single target sequence and the complementary regions are at least 200-30,000 nts apart on the target sequence, (iii) primer annealing sites at the 5′ end of the pre-LASSO probes and between the ligation arm and extension arm sequences, and (iv) a fusion overlapping sequence at the 3′ end of the pre-LASSO probes, wherein the plurality of pre-LASSO probes comprises probes with sequences complementary to 10 or more different target sequences, wherein all or a subset of the pre-probes have the same primer annealing site sequences and fusion overlapping sequences.

5. A method of generating the plurality of oligonucleotides of claim 3, comprising:
(i) providing a plurality of pre-LASSO probes, wherein the pre-LASSO probes are synthetically generated, comprising (i) a ligation arm sequence of 15-80 bp long that is complementary to a 5′ region of a target sequence, (ii) an extension arm sequence of 15-80 bp that is complementary to a 3′ region of a target sequence, wherein the ligation arm and extension arm sequences are complementary to 5′ and 3′ regions of a single target sequence and the complementary regions are at least 200-30,000 nts apart on the target sequence, (iii) primer annealing sites at the 5′ end of the pre-LASSO probes and between the ligation arm and extension arm sequences, and (iv) a fusion overlapping sequence at the 3′ end of the pre-LASSO probes, wherein the plurality of pre-LASSO probes comprises probes with sequences complementary to 10 or more different target sequences, wherein all or a subset of the pre-probes have the same primer annealing site sequences and fusion overlapping sequences;
(ii) contacting the plurality of pre-LASSO probes with a plurality of Long Adapter Oligonucleotides in a single reaction sample, wherein the Long Adapter Oligonucleotides comprise a sequence of 200 to 2500 nt comprising a fusion overlapping sequence that is complementary to the fusion overlapping sequence on the pre-LASSO probes, a primer annealing site of 15-80 nts, optionally one or more restriction enzyme recognition sites and a long adapter sequence, under conditions to allow hybridization of the fusion overlapping sequences of the long adapters to the pre-probes at the fusion overlapping sequence;
(iii) using overlap-extension polymerase chain reaction (PCR) to extend the hybridized regions to generate a double stranded linear DNA fragment;
(iv) digesting the double-stranded linear DNA fragment to create complementary overhangs or blunt ends to allow circularization of the double-stranded DNA fragment;
(v) circularizing the double-stranded DNA fragment by enzymatic and/or chemical ligation; and
(vi) using inverted PCR with primers that bind to the primer annealing sites between the ligation arm and extension arm sequences to create linear double-stranded DNA fragments with the primer annealing sites at the 5′ and 3′ ends of linear double-stranded DNA fragments; and
(viii) removing all or part of the primer annealing sites from the 5′ and 3′ ends of linear oligonucleotides by restriction digestion and/or glycosylase digestion.

6. A method of creating a library of 10 or more different target sequences from a sample, the method comprising,
contacting the sample with the plurality of the oligonucleotides of claim 3 in a single reaction sample, wherein the plurality includes oligonucleotides with sequences complementary to the different target sequences, under conditions sufficient to allow hybridization of the ligation arm and extension arm sequences of the oligonucleotides to target sequences in the sample;
gap filling using polymerase and ligase to copy the target sequence between the ligation arm and extension arm and ligate the resulting molecule, to create circular single-stranded DNA fragments comprising the target sequences;
purifying the circular single-stranded DNA fragments comprising the target sequences, optionally by digesting linear DNA in the sample; and
amplifying the circular single-stranded DNA fragments comprising the target sequences, thereby amplifying the target sequences.

7. The method of claim 6, wherein the target sequences are at least 200-500 base pairs (bp) long.

8. The method of claim 7, wherein the target sequences are at least 200-30,000 long.

9. The method of claim 6, wherein gap filling using polymerase and ligase comprises using 0.03-0.05 U/μl polymerase and 0.02-0.1 U/μl thermostable ligase.

10. The method of claim 6, wherein hybridization of the ligation arm and extension arm sequences of the oligonucleotides to target sequences, and gap filling were performed at 55-75° C.

11. The method of claim 1, wherein the target sequences comprise 10,000 or more different target sequences.

12. The method of claim 1, wherein the sample is a genomic DNA (gDNA) sample.

13. The method of claim 1, wherein the sample comprises cDNA.

14. A library of target sequences created by the method of claim 6.

15. A kit comprising the plurality of the oligonucleotides of claim 3, polymerase, and ligase.
